(19) United States
(12) Patent Application Publication
(10) Pub. No.: US 2024/0058317 A1
(43) Pub. Date: Feb. 22, 2024

(54) LOMITAPIDE FOR USE IN METHODS OF TREATING HYPERLIPIDEMIA AND HYPERCHOLESTEROLEMIA IN PEDIATRIC PATIENTS

(71) Applicant: Amryt Pharmaceuticals Inc., Boston, MA (US)

(72) Inventors: Ruth NALLEN, Dublin (IE); Tracy CUNNINGHAM, Dublin (IE); Mark SUMERAY, Zug (CH)

(21) Appl. No.: 18/489,542

(22) Filed: Oct. 18, 2023

Related U.S. Application Data
(63) Continuation of application No. 18/018,031, filed on Jan. 25, 2023.
(60) Provisional application No. 63/058,211, filed on Jul. 29, 2020.

(51) Int. Cl. A61K 31/4468; A61P 3/06

(52) U.S. Cl. CPC A61K 31/4468; A61P 3/06

(57) ABSTRACT

Provided herein are methods of treating hyperlipidemia or hypercholesterolemia in pediatric patients with lomitapide or a pharmaceutically acceptable salt thereof.

### CROSS-REFERENCE TO RELATED APPLICATIONS

This application claims the benefit of priority to U.S. Application Ser. No. 63/058,211, filed Jul. 29, 2020, the contents of which is hereby incorporated by reference in its entirety for all purposes.

### BACKGROUND

Homozygous familial hypercholesterolaemia (HoFH) is a rare and life-threatening inherited disorder of lipid metabolism with an estimated prevalence of 1 per 160,000 to 300,000 in the European population. The Consensus Panel on Familial Hypercholesterolaemia (FH) of the European Atherosclerosis Society (EAS) recommends the following criteria for the diagnosis of HoFH:
i) genetic confirmation of 2 mutant alleles at the LDL receptor (LDLR), apolipoprotein B (apo B), proprotein convertase subtilisin/kexin type 9 (PCSK9), or LDL-receptor adapter protein 1 (LDLRAP1) gene locus or
ii) an untreated low-density lipoprotein cholesterol (LDL-C)>500 mg/dL (13 mmol/L) and treated LDL-C≥300 mg/dL (8 mmol/L), respectively together with either cutaneous or tendon xanthomas before the age of 10 years or untreated LDL-C levels consistent with heterozygous FH in both parents.

Significantly elevated LDL-C levels lead to premature, severe, and progressive atherosclerosis and development of early cardiovascular disease (CVD). The average age of death is 18 years if untreated and effective lipid-lowering therapy (LLT) greatly improves survival in HoFH. Based on the evidence that treatment can delay the onset of CVD, primary prevention is suggested to start as early as possible and mainly consists of lowering LDL-C levels to <135 mg/dL (<3.5 mmol/L) in pediatric HoFH patients and to <100 mg/dL (<2.5 mmol/L) in adults. Secondary prevention comprises a decrease of LDL-C levels to <70 mg/dL (<1.8 mmol/L) in adults with CVD as recommended by the EAS.

A number of treatments are currently available for lowering serum cholesterol and triglycerkles. However, each has its own drawbacks and limitations in terms of efficacy, side-effects and qualifying patient population.

Thus, there is a need for safe, early and effective treatment of pediatric patients with HoFH.

### BRIEF DESCRIPTION OF THE DRAWINGS

FIG. 1 shows the mean lomitapide pediatric starting dose (mg) based on PBPK modeling and allometric scaling.

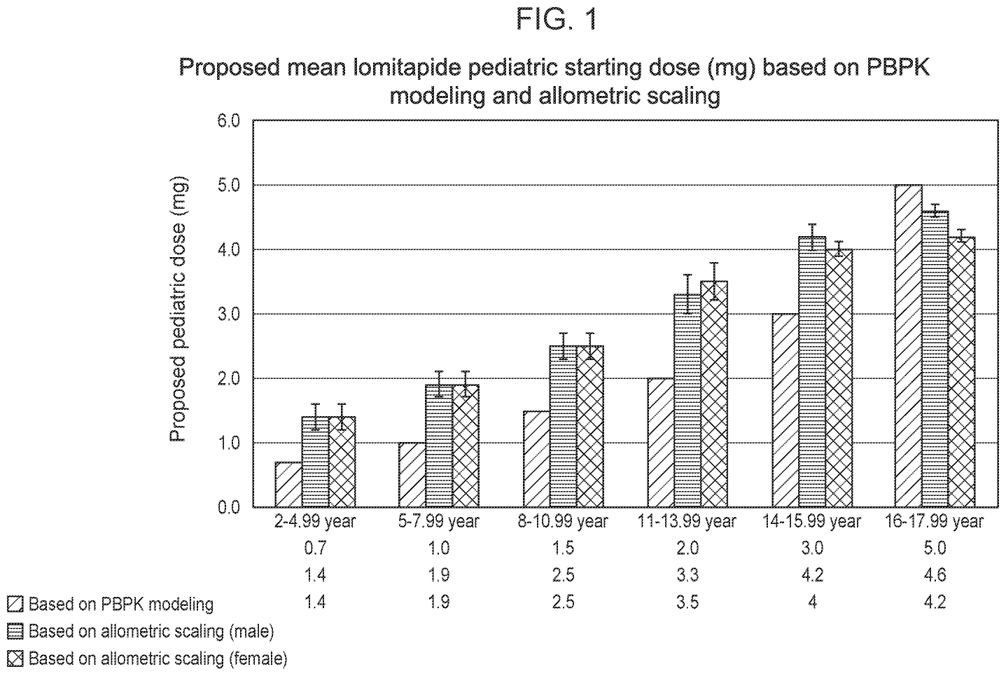

FIG. 2 shows the design of the clinical study in Example 2.

### SUMMARY

In embodiments, the present disclosure provides, a method of safely administering lomitapide, or a pharmaceutically acceptable salt thereof, to a pediatric patient comprising:
(a) administering a first daily dose of about 0.5 mg to about 2.5 mg of lomitapide, or a pharmaceutically acceptable salt thereof to a patient aged 5 to 15 years; and
(b) administering a first daily dose of about 2.5 mg to about 7.5 mg of lomitapide, or a pharmaceutically acceptable salt thereof to a patient aged 16 to 17 years,
wherein the first daily dose is administered to the patient for about 1 to 4 weeks.

In embodiments, the present disclosure provides a method of treating hyperlipidemia or hypercholesterolemia in a pediatric patient, comprising:
(a) administering a first daily dose of about 0.5 mg to about 2.5 mg of lomitapide, or a pharmaceutically acceptable salt thereof to a patient's age is 5 to 15 years;
(b) administering a first daily dose of about 2.5 mg to about 7.5 mg of lomitapide, or a pharmaceutically acceptable salt thereof to a patient's age is 16 to 17 years,
wherein the first daily dose is administered to the patient for about I to 4 weeks.

In embodiments, provided herein is a method of treating hyperlipidemia or hypercholesterolemia, comprising:
administering a first daily dose of about 0.5 mg to about 2.5 mg of lomitapide, or a pharmaceutically acceptable salt thereof to a pediatric patient age 5 to 10 years, wherein the first daily dose is administered to the patio nt for about 1 to 4 weeks.

In embodiments, provided herein is a method of treating hyperlipidemia or hypercholesterolemia, comprising:
administering a first daily dose of about 0.5 mg to about 2.5 mg of lomitapide, or a pharmaceutically acceptable salt thereof to a pediatric patient age 11 to 15 years, wherein the first daily dose is administered to the patkmt for about I to 4 weeks.

In embodiments, the present disclosure provides a method of treating hyperlipidemia or hypercholesterolemia, comprising:
administering a first daily dose of about 2.5 mg to about 7.5 mg of lomitapide, or a pharmaceutically acceptable salt thereof to a pediatric patient age 16 to 17 years, wherein the first daily dose is administered to the patient for about 1 to 4 weeks.

### Definitions

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The terms “effective amount” and “therapeutically effective amount” are used interchangeably in this disclosure and refer to an amount of a compound, or a salt, solvate or ester thereof, that, when administered to a patient, is capable of performing the intended result. For example, an effective amount of lomitapide is that amount which is required to reduce at least one symptom of HoFH in a patient, e.g. the amount required to reduce the patient's low-density lipoprotein cholesterol (LDL-C). The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pediatric patient” as used herein refers to a patient less than 18 years of age.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethnesulfonic, sulfanilic, cyclohexylaminosulfonic, algenic, 3-hydroxybutyric, galactaric and galacturonic acid.

The term “treating” as used herein with regard to a patient, refers to improving at least one symptom of the patient's disorder. Treating can be improving, or at least partially ameliorating a disorder.

The term “therapeutic effect” as used herein refers to a desired or beneficial effect provided by the method and/or the composition. For example, the method for treating HoFH provides a therapeutic effect when the method reduces at least one symptom of HoFH, e.g., reduce low-density lipoprotein cholesterol (LDL-C), in a patient.

### DETAILED DESCRIPTION

### Lomitapide

Lomitapide is a first in class oral, selective inhibitor of microsomal transfer protein (MTP), an intracellular lipid-transfer protein that is found in the lumen of the endoplasmic reticulum and is responsible for binding and shuttling individual lipid molecules between membranes. MTP plays a key role in the assembly of apo B containing lipoproteins in the liver and intestines. Inhibition of MTP reduces lipoprotein secretion and circulating concentrations of lipoprotein-borne lipids including cholesterol and triglycerides.

The chemical name of lomitapide is N-(2,2,2-trifluoroethyl)-9-[4-[4-[[[4′(trifluoromethyl)[1,1′-biphenyl]-2-yl]carbonyl]amino]1-piperidinyl]butyl]-9H-fluorene-9-carboxamide. Its structural formula is:

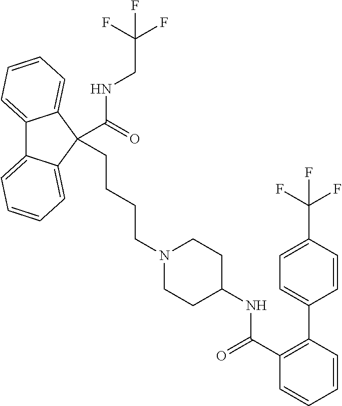 [SMILES: O=C(NC1CCN(CCCCC2(C(=O)NCC(F)(F)F)c3ccccc3-c3ccccc32)CC1)c1ccccc1-c1ccc(C(F)(F)F)cc1]

Lomitapide and other inhibitors of MTP-mediated neutral lipid transfer activity are described, for example, in U.S. Pat. Nos. 5,789,197, 5,883,109, 6,066,653, and 6,492,365, each of which is incorporated herein by reference in its entirety. MTP inhibitors are described throughout U.S. Pat. No. 6,066,653, in particular in columns 3-28. Lomitapide and methods for its use are described, for example, in U.S. Pat. No. 7,932,268; 8,618,135; 9,265,758; 9,364,470; 9,433,617; 9,861,622, 10,016,404, and 10,555,938 each of which is incorporated by reference herein in its entirety. See also U.S. Pat. No. 10,213,419 the entirety of which is incorporated herein by reference.

The European Commission (EC) granted authorization for lomitapide under the trade name ‘Lojuxta®’ in July 2013. Lomitapide is a “lipid modifying agent” according to the Anatomical Therapeutic Chemical (ATC) Classification System (ATC code C10AX12). It is indicated as an adjunct to a low-fat diet and other lipid-lowering medicinal products with or without LA in adult patients with HoFH. Genetic confirmation of HoFH should be obtained whenever possible. Other forms of primary hyperlipoproteinaemia and secondary causes of hypercholesterolaemia (e.g., nephrotic syndrome, hypothyroidism) must be excluded.

However, Lojuxta is not approved in pediatric patients (i.e., in patients less than 18 years of age) because the safety and efficacy of lomitapide in this sensitive population has not been established.

In formulating the compositions, lomitapide or a pharmaceutically acceptable salt thereof, in the amounts described herein, may be compounded according to accepted pharmaceutical practice with a physiologically acceptable vehicle, carrier, excipient, binder, preservative, stabilizer, flavor, etc., in the particular type of unit dosage form. Such dosage forms can be administered to the patient on a regimen of one to four doses per day.

In one aspect, the disclosure provides tablets containing a lomitapide composition as described herein. A tablet may be made by compression or molding, optionally with one or more accessory ingredients. Compressed tablets may be prepared using binder (for example, microcrystalline cellulose, or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art. The disclosed excipients may serve more than one function. For example, fillers or binders may also be disintegrants, glidants, anti-adherents, lubricants, sweeteners and the like

Liquid formulations can also be prepared by dissolving or suspending one or the combination of active substances in a conventional liquid vehicle acceptable for pharmaceutical administration so as to provide the desired dosage in one to four teaspoonsfuls.

Dosage units including tablets, capsules and caplets, of various sizes can be prepared, e.g., of about 2 to 10000 mg in total weight, containing one or both of the active substances in the ranges described above, with the remainder being a physiologically acceptable carrier of other materials according to accepted pharmaceutical practice. These tablets can, of course, be scored to provide for fractional doses. Gelatin capsules can be similarly formulated. For example, in some embodiments a scored tablet may provide the dosage unit. Under the direction of a physician or other medical professional, the subject may be directed to take one portion of the dosage unit, wherein the one portion will provide the desired dosage level for given interval. At the following interval, the patient may be instructed to take two or more portions of the dosage unit wherein the two or more portions will provide the desired dosage level for that interval.

Formulations of lomitapide are commercially available, for example, as Juxtapid capsules. Each Juxtapid capsule contains lomitapide mesylate equivalent to 5, 10, 20, or 30 mg lomitapide free base and the following inactive ingredients: pregelatinized starch, sodium starch glycolate, microcrystalline cellulose, lactose monohydrate, silicon dioxide and magnesium stearate. The capsule shells of all strengths contain gelatin and titanium dioxide; the 5 mg, 10 mg and 30 mg capsules also contain red iron oxide; and the 30 mg capsules also contain yellow iron oxide. The imprinting ink contains shellac, black iron oxide, and propylene glycol. However, the scope of the present disclosure is not limited to dosage strengths of Juxtapid presently available commercially, and includes capsules containing lomitapide mesylate (or other pharmaceutically acceptable salts of lomitapide) equivalent to 5, 10, 20, 30, 40, or 60 mg lomitapide free base.

### Methods of the Present Disclosure

The present disclosure provides methods of safely administering lomitapide to pediatric HoFH patients. It will be understood that in embodiments of the methods provided herein, the patient is a human.

The disclosure provides methods for treating hyperlipidemia or hypercholesterolemia (e.g., Homozygous Familial Hypercholesterolaemia (HoFH)) by administering an effective and tolerable amount of lomitapide or a pharmaceutically acceptable salt thereof, to a pediatric patient in need thereof. An effective amount is an amount sufficient significantly reduce hyperlipidemia or hypercholesterolemia (e.g., HoFH) symptoms or to alleviate those symptoms. Formulations employed in the present methods can incorporate lomitapide or a pharmaceutically acceptable salt thereof into a formulation such that the formulation provides therapeutically effective blood plasma levels of lomitapide or a pharmaceutically acceptable salt thereof for the treatment of hyperlipidemia or hypercholesterolemia (e.g., HoFH).

In some embodiments, a therapeutically effective dose is achieved by starting the patient on an initial daily dose and titrating to an efficacious and tolerated dose by gradually modifying (e.g., increasing or decreasing) the daily administered amount of lomitapide or a pharmaceutically acceptable salt thereof until a dose that is effective and tolerated is achieved (e.g., the patient with hyperlipidemia or hypercholesterolemia (Homozygous Familial Hypercholesterolaemia (HoFH),) is treated). In some embodiments, the efficacious dose is a dose that improves at least one symptom of the patient's Homozygous Familial Hypercholesterolaemia (HoFH), or hyperlipidemia or hypercholesterolemia. In some embodiments, the efficacious dose is a dose that reduces the patient's LDL-C to <135 mg/dL (3.5 mmol/L).

In some embodiments, the lomitapide or a pharmaceutically acceptable salt thereof is administered in escalating doses. In some embodiments, the escalating doses comprise at least a first dose level and a second dose level. In some embodiments, the escalating doses comprise at least a first dose level, a second dose level, and a third dose level. In some embodiments, the escalating doses further comprise a fourth dose level. In some embodiments, the escalating doses further comprise a fifth dose level. In some embodiments, the escalating doses comprise a first dose level, a second dose level, a third dose level, a fourth dose level and a fifth dose level. In some embodiments, six, seven, eight, nine and ten dose levels are contemplated.

In some embodiments, each dose level is no more than 60% of the immediately following dose level. In embodiments, each dose level is no more than 50% of the immediately following dose level. In embodiments, each dose level is no more than 40% of the immediately following dose level. In some embodiments, each dose level is no more than 33% of the immediately following dose level. In some embodiments, each dose level is no more than 20% of the immediately following dose level. In some embodiments, dose levels are separated by ½ log units. In some embodiments, dose levels are separated by 1 log unit.

In one aspect, the present disclosure provides a method of safely administering lomitapide, or a pharmaceutically acceptable salt thereof, to a pediatric patient comprising:
(a) administering a first daily dose of about 0.5 mg to about 2.5 mg of lomitapide, or a pharmaceutically acceptable salt thereof to a patient aged 5 to 15 years; and
(b) administering a first daily dose of about 2.5 mg to about 7.5 mg of lomitapide, or a pharmaceutically acceptable salt thereof to a patient aged 16 to 17 years,
wherein the first daily dose is administered to the patient hr about 1 to 4 weeks.

In some embodiments, the method of safely administering lomitapide, or a pharmaceutically acceptable salt thereof, to a pediatric patient comprises administering a first daily dose of about 2 mg of lomitapide, or a pharmaceutically acceptable salt thereof to a patient aged 5 to 15 years.

In some embodiments, the method of safely administering lomitapide, or a pharmaceutically acceptable salt thereof, to a pediatric patient comprises administering a first daily dose of about 5 mg of lomitapide, or a pharmaceutically acceptable salt thereof to a patient aged 16 to 17 years.

In one aspect, the present disclosure provides a method of treating hyperlipidemia or hypercholesterolemia in a pediatric patient, comprising:
(a) administering a first daily dose of about 0.5 mg to about 2.5 mg of lomitapide, or a pharmaceutically acceptable salt thereof to a patient's age is 5 to 15 years;
(b) administering a first daily dose of about 2.5 mg to about 7.5 mg of lomitapide, or a pharmaceutically acceptable salt thereof to a patient's age is 16 to 17 years,
wherein the first daily dose is administered to the patient for about 1 to 4 weeks.

In some embodiments, the method of treating hyperlipidemia or hypercholesterolemia in a pediatric patient comprises administering a first daily dose of about 2 mg of lomitapide, or a pharmaceutically acceptable salt thereof to a patient aged 5 to 15 years.

In some embodiments, the method of treating hyperlipidemia or hypercholesterolemia in a pediatric patient comprises administering a first daily dose of about 5 mg of lomitapide, or a pharmaceutically acceptable salt thereof to a patient aged 16 to 17 years.

In one aspect, the present disclosure provides a method of treating hyperlipidemia or hypercholesterolemia, comprising:
administering a first daily dose of about 0.5 mg to about 2.5 mg of lomitapide, or a pharmaceutically acceptable salt thereof to a pediatric patient age 5 to 10 years, wherein the first daily dose is administered to the patient for about 1 to 4 weeks.

In one aspect, the present disclosure provides a method of treating hyperlipidemia or hypercholesterolemia, comprising:
administering a first daily dose of about 0.5 mg to about 2.5 mg of lomitapide, or a pharmaceutically acceptable salt thereof to a pediatric patient age 11 to 15 years, wherein the first daily dose is administered to the patient for about 1 to 4 weeks.

In one aspect, the present disclosure provides a method of treating hyperlipidemia or hypercholesterolemia, comprising:

administering a first daily dose of about 2.5 mg to about 7.5 mg of lomitapide, or a pharmaceutically acceptable salt thereof to a pediatric patient age 16 to 17 years, wherein the first daily dose is administered to the patient for about 1 to 4 weeks.

In some embodiments, the first daily dose is administered to the patient for about 4 weeks. In embodiments, the first daily dose is administered for about 1 week to about 6 weeks, including about 1 week, about 2 weeks, about 3 weeks, about 4 weeks, about 5 weeks, or about 6 weeks, including all values and subranges therebetween.

In some embodiments each dose level of lomitapide or a pharmaceutically acceptable salt thereof is administered to the subject for from 2 days to 30 weeks, or more. In some embodiments each dose level is administered to the subject for from about 1 week to about 12 weeks. In some embodiments, each dose level is administered to the subject for about 1 week to about 5 weeks. In some embodiments each dose level is administered to the subject from about 1 to about 4 weeks. In some embodiments each dose level is administered to the subject from about 1 to about 3 weeks. In some embodiments each dose level is administered to the subject from about 1 to about 2 weeks.

In some embodiments, lomitapide or a pharmaceutically acceptable salt is administered. In some embodiments, lomitapide mesylate is administered. In some embodiments, lomitapide or a pharmaceutically acceptable salt thereof is administered orally.

In some embodiments, lomitapide or a pharmaceutically acceptable salt thereof is administered in a daily dose of about 0.1 mg to about 100 mg to a pediatric patients with hyperlipidemia or hypercholesterolemia (Homozygous Familial Hypercholesterolaemia (HoFH)) in need thereof, including about 0.1 mg, about 0.2 mg, about 0.3 mg, about 0.4 mg, about 0.5 mg, about 1 mg, about 2 mg, about 2.5 mg, about 5 mg, about 7.5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg to about 100 mg, including any subrange or value therebetween. In some embodiments, about 2 mg to about 60 mg of lomitapide or a pharmaceutically acceptable salt thereof is administered on a daily basis. In some embodiments, about 2 mg to about 20 mg of lomitapide or a pharmaceutically acceptable salt thereof is administered on a daily basis. In some embodiments, about 2 mg to about 40 mg of lomitapide or a pharmaceutically acceptable salt thereof is administered on a daily basis.

In some embodiments, the pediatric patient in need of hyperlipidemia or hypercholesterolemia (e.g.,Homozygous Familial Hypercholesterolaemia (HoFH)) treatment is administered a daily dose of 2 mg of lomitapide or a pharmaceutically acceptable salt thereof in the first dosing period, a daily dose of 2 mg of lomitapide or a pharmaceutically acceptable salt thereof in the second dosing period, and a daily dose of 5 mg of lomitapide or a pharmaceutically acceptable salt thereof in the third dosing period, a daily dose of 10 mg of lomitapide or a pharmaceutically acceptable salt thereof in the fourth dosing period, and a daily dose of 20 mg of lomitapide or a pharmaceutically acceptable salt thereof in the fifth dosing period.

In some embodiments, the pediatric patient in need of hyperlipidemia or hypercholesterolemia(e.g., Homozygous Familial Hypercholesterolaemia (HoFH)) treatment is administered a daily dose of 2 mg of lomitapide or a pharmaceutically acceptable salt thereof in the first dosing period, a daily dose of 5 mg of lomitapide or a pharmaceutically acceptable salt thereof in the second dosing period, and a daily dose of 10 mg of lomitapide or a pharmaceutically acceptable salt thereof in the third dosing period, a daily dose of 20 mg of lomitapide or a pharmaceutically acceptable salt thereof in the fourth dosing period, and a daily dose of 40 mg of lomitapide or a pharmaceutically acceptable salt thereof in the fifth dosing period.

In some embodiments, the pediatric patient in need of hyperlipidemia or hypercholesterolemia (e.g., Homozygous Familial Hypercholesterolaemia (HoFH)) treatment is administered a daily dose of 5 mg of lomitapide or a pharmaceutically acceptable salt thereof in the first dosing period, a daily dose of 10 mg of lomitapide or a pharmaceutically acceptable salt thereof in the second dosing period, and a daily dose of 20 mg of lomitapide or a pharmaceutically acceptable salt thereof in the third dosing period, a daily dose of 40 mg of lomitapide or a pharmaceutically acceptable salt thereof in the fourth dosing period, and a daily dose of 60 mg of lomitapide or a pharmaceutically acceptable salt thereof in the fifth dosing period.

In some embodiments, the first dosing period, second dosing period, or third dosing period is about or at least about 1-8 weeks, including about or at least about 1 week, about or at least about 2 weeks, about or at least about 3 weeks, about or at least about 4 weeks, about or at least about 5 weeks, about or at least about 6 weeks, about or at least about 7 weeks, or about or at least about 8 weeks, including all subranges and values therebetween.

In some embodiments, the patient's age is from 5 to 10 years, from 11 to 15 years, or from 16 to 17 years.

In some embodiments of any of the methods disclosed herein, the lomitapide is provided to the patient as an adjunct to a low-fat diet and other lipid-lowering treatments, including LDL apheresis. In some embodiments, concomitant lipid-lowering treatments include one or more of the following: statins, ezetimibe, nicotinic acid, bile acid sequestrant, fibrate, and apheresis.

In some embodiments, the low-fat diet comprises a diet wherein less than about 30% of patient's total calories are from fat, less than about 20% of patient's total calories are from fat, less than about 15% of patient's total calories are from fat, or less than about 10% of patient's total calories are from fat. In some embodiments, the low-fat diet comprises a diet wherein less than about 20% of patient's total calories are from fat.

In some embodiments, patients receiving lipid lowering therapies during treatment with lomitapide are administered dietary supplements that provided approximately 400 international units vitamin E, 210 mg alpha-linolenic acid (ALA), 200 mg linoleic acid, 110 mg eicosapentaenoic acid (EPA), and 80 mg docosahexaenoic acid (DHA) per day.

In some embodiments of any of the methods disclosed herein, administration of lomitapide to a pediatric patient reduces one or more of the following: low-density lipoprotein cholesterol (LDL-C), total cholesterol (TC), apolipoprotein B (apo B), and non-high density lipoprotein cholesterol (non-HDL-C) in patients with homozygous familial hypercholesterolemia (HoFH). In some embodiments, the lomitapide is provided to the patient as an adjunct to a low-fat diet and other lipid-lowering treatments, including LDL apheresis.

In some embodiments, administration of lomitapide to a pediatric patient reduces one or more of the following: low-density lipoprotein cholesterol (LDL-C), total cholesterol (TC), apolipoprotein B (apo B), and non-high density lipoprotein cholesterol (non-HDL-C) in patients with homozygous familial hypercholesterolemia (HoFH).

In some embodiments, administration of lomitapide to a pediatric patient as an adjunct to a low-fat diet and other lipid-lowering treatments (e.g., as disclosed herein) according to any of the methods disclosed herein reduces one or more of the following: low-density lipoprotein cholesterol (LDL-C), total cholesterol (TC), apolipoprotein B (apo B), and non-high density lipoprotein cholesterol (non-HDL-C) by about or at least about 10% to about or at least about 90% or more, including about or at least about 10%, about or at least about 20%, about or at least about 30%, about or at least about 40%, about or at least about 50%, about or at least about 60%, about or at least about 70%, about or at least about 80%, about or at least about 90%, or more, including all values and subranges therebetween.

In embodiments of any of the methods disclosed herein, the maximum dose in a pediatric patient with Child-Pugh A aged from 5 to 10 years is 10 mg.

In embodiments of any of the methods disclosed herein, the maximum dose in a pediatric patient with Child-Pugh A aged from 11 to 15 years is 20 mg.

In embodiments of any of the methods disclosed herein, the maximum dose in a pediatric patient with Child-Pugh A aged from 11 to 15 years is 40 mg.

In some embodiments, the patient's age is from 5 to 10 years and the daily dose of lomitapide in the first dosing period is 2 mg, the daily dose of lomitapide in the second dosing period is 2 mg, and the daily dose of lomitapide in the third dosing period is 5 mg, the daily dose of lomitapide in the fourth dosing period is 10 mg. In some embodiments, the first dosing period is about or at least about 4 weeks, the second dosing period is about or at least about 4 weeks, and the third dosing period is about or at least about 4 weeks, the fourth dosing period is about or at least about 4 weeks, and the fifth dosing period is about or at least about 4 weeks. In some embodiments, the first, second, third, fourth, and fifth dosing periods are each about 4 weeks ±3 days.

In some embodiments, the patient's age is from 11 to 15 years and the daily dose of lomitapide in the first dosing period is 2 mg, the daily dose of lomitapide in the second dosing period is 5 mg, the daily dose of lomitapide in the third dosing period is 10 mg, the daily dose of lomitapide is about 20 mg in the fourth dosing period, the daily dose is about 40 mg in the fifth dosing period. In some embodiments, the first dosing period is about or at least about 4 weeks, the second dosing period is about or at least about 4 weeks, and the third dosing period is about or at least about 4 weeks, the fourth dosing period is about or at least about 4 weeks, and the fifth dosing period is about and at least about 4 weeks. In some embodiments, the first, second, third, fourth, and fifth dosing periods are each about 4 weeks ±3 days.

In some embodiments, the patient's age is 16 to 17 years and the daily dose of lomitapide in the first dosing period is about 5 mg, the daily dose of lomitapide in the second dosing period is about 10 mg, the daily dose of lomitapide in the third dosing period is about 20 mg, the daily dose of lomitapide in the fourth dosing period is about 40 mg, and the daily dose of lomitapide in the fifth dosing period is about 60 mg. In some embodiments, the first dosing period is about or at least about 4 weeks, the second dosing period is about or at least about 4 weeks, and the third dosing period is about or at least about 4 weeks, the fourth dosing period is about or at least about 4 weeks, and the fifth dosing period is about or at least about 4 weeks. In some embodiments, the first, second, third, fourth, and fifth dosing periods are each about 4 weeks ±3 days.

In some embodiments the daily dose is administered as a single dose or divided into 2 or 3 equal or unequal doses. In some embodiments, the lomitapide is administered once-daily at bedtime.

In some embodiments, lomitapide mesylate in an amount equivalent to about 2-60 mg of the lomitapide free base is administered. In some embodiments, lomitapide mesylate in an amount equivalent to about 2mg of the lomitapide free base is administered. In some embodiments, lomitapide mesylate in an amount equivalent to about 2.5 mg of the lomitapide free base is administered. In some embodiments, lomitapide mesylate in an amount equivalent to about 5 mg of the lomitapide free base is administered. In some embodiments, lomitapide mesylate in an amount equivalent to about 10 mg of the lomitapide free base is administered. In some embodiments, lomitapide mesylate in an amount equivalent to about 30 mg of the lomitapide free base is administered. In some embodiments, lomitapide mesylate in an amount equivalent to about 40 mg of the lomitapide free base is administered. In some embodiments, lomitapide mesylate in an amount equivalent to about 60 mg of the lomitapide free base is administered.

In some embodiments, the patient has homozygous familial hypercholesterolemia.

### Kits

In some embodiments, the present disclosure provides kits for use in treating hyperlipidemia or hypercholesterolemia (e.g., Homozygous Familial Hypercholesterolaemia (HoFH)) in a patient in need thereof. Such kits comprise lomitapide or a pharmaceutically salt thereof The kits of the present disclosure may be used for administering lomitapide or a pharmaceutically acceptable salt thereof at different dosage intervals, or for titrating the dose of lomitapide or a pharmaceutically acceptable salt thereof according to methods described herein. For example, the present disclosure provides kits for treating hyperlipidemia or hypercholesterolemia (e.g., Homozygous Familial Hypercholesterolaemia (HoFH)) in a pediatric subject, comprising at least three sets of pharmaceutical dosage units; and instructions for use.

In some embodiments, the kits of the present disclosure may comprise directions for administration. For example, the kit can include instructions to administer lomitapide or a pharmaceutically acceptable salt thereof in a suitable manner to perform the methods described herein, e.g., in a suitable dose, dosage form, dosing intervals (e.g., as described herein). In some embodiments, the informational material can include instructions to administer the lomitapide or a pharmaceutically acceptable salt thereof to a pediatric patient with hyperlipidemia or hypercholesterolemia (e.g., Homozygous Familial Hypercholesterolaemia (HoFH)).

The kit can include one or more containers for lomitapide or a pharmaceutically salt thereof as described herein. In some embodiments, the kit contains separate containers, dividers or compartments for the composition and informational material. For example, the composition can be contained in a bottle, vial, or syringe. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a composition described herein. For example, the kit can include a plurality of syringes, ampules, or foil packets each containing a single unit dose of a composition described herein. An example of such a kit is a blister pack, as typically used for the packaging of tablets, capsules and the like. The containers of the kits can be air tight, waterproof (e.g., impermeable to changes in moisture or evaporation), and/or light-tight.

In some embodiments, provided herein is a kit for safely administering lomitapide, or a pharmaceutically acceptable salt thereof, to a pediatric patient aged 5 to 15 comprising: a first daily dose of about 0.5 mg to about 2.5 mg of lomitapide, or a pharmaceutically acceptable salt thereof.

In some embodiments, provided herein is a kit for safely administering lomitapide, or a pharmaceutically acceptable salt thereof, to a pediatric patient aged 16 to 17 years, comprising a first daily dose of about 2.5 mg to about 7.5 mg of lomitapide, or a pharmaceutically acceptable salt thereof.

In some embodiments, provided herein is a kit for treating hyperlipidemia or hypercholesterolemia (e.g., homozygous familial hypercholesterolemia) in a pediatric patient aged 5 to 15 years comprising: a first daily dose of about 0.5 mg to about 2.5 mg of lomitapide, or a pharmaceutically acceptable salt thereof.

In some embodiments, provided herein is a kit for treating hyperlipidemia or hypercholesterolemia (e.g., homozygous familial hypercholesterolemia) in a pediatric patient aged 16 to 17 years, comprising a first daily dose of about 2.5 mg to about 7.5 mg of lomitapide, or a pharmaceutically acceptable salt thereof.

In some embodiments, provided herein is a kit for treating hyperlipidemia or hypercholesterolemia (e.g., homozygous familial hypercholesterolemia) in a pediatric patient aged 5 to 10 years comprising: a first daily dose of about 0.5 mg to about 2.5 mg of lomitapide, or a pharmaceutically acceptable salt thereof.

In some embodiments, provided herein is a kit for treating hyperlipidemia or hypercholesterolemia (e.g., homozygous familial hypercholesterolemia) in a pediatric patient aged 11 to 15 years comprising: a first daily dose of about 0.5 mg to about 2.5 mg of lomitapide, or a pharmaceutically acceptable salt thereof.

In some embodiments, provided herein is a kit for treating hyperlipidemia or hypercholesterolemia in a pediatric patient aged 16 to 17 years comprising a first daily dose of about 2.5 mg to about 7.5 mg of lomitapide, or a pharmaceutically acceptable salt thereof.

In some embodiments of the kits provided herein, the kit contains a first daily dose to be administered to the pediatric patient for about 1 to 4 weeks, including about 1 week, about 2 weeks, about 3 weeks and about 4 weeks. In some embodiments, the kit contains a first daily dose to be administered to the pediatric patient for about 4 weeks.

In some embodiments of the kits provided herein for treating hyperlipidemia or hypercholesterolemia (e.g., Homozygous Familial Hypercholesterolaemia (HoFH)) in a pediatric patient, the kit contains a daily dose of 2 mg of lomitapide or a pharmaceutically acceptable salt thereof to be administered in a first dosing period, a daily dose of 2 mg of lomitapide or a pharmaceutically acceptable salt thereof to be administered in a second dosing period, a daily dose of 5 mg of lomitapide or a pharmaceutically acceptable salt thereof to be administered in a third dosing period, a daily dose of 10 mg of lomitapide or a pharmaceutically acceptable salt thereof to be administered in a fourth dosing period, and a daily dose of 20 mg of lomitapide or a pharmaceutically acceptable salt thereof to be administered in a fifth dosing period.

In some embodiments of the kits provided herein for treating hyperlipidemia or hypercholesterolemia (e.g., Homozygous Familial Hypercholesterolaemia (HoFH)) in a pediatric patient, the kit contains a daily dose of 2 mg of lomitapide or a pharmaceutically acceptable salt thereof to be administered in a first dosing period, a daily dose of 5 mg of lomitapide or a pharmaceutically acceptable salt thereof to be administered in a second dosing period, a daily dose of 10 mg of lomitapide or a pharmaceutically acceptable salt thereof to be administered in a third dosing period, a daily dose of 20 mg of lomitapide or a pharmaceutically acceptable salt thereof to be administered in a fourth dosing period, and a daily dose of 40 mg of lomitapide or a pharmaceutically acceptable salt thereof to be administered in a fifth dosing period.

In some embodiments of the kits provided herein for treating hyperlipidemia or hypercholesterolemia (e.g., Homozygous Familial Hypercholesterolaemia (HoFH)) in a pediatric patient, the kit contains a daily dose of 5 mg of lomitapide or a pharmaceutically acceptable salt thereof to be administered in a first dosing period, a daily dose of 10 mg of lomitapide or a pharmaceutically acceptable salt thereof to be administered in a second dosing period, a daily dose of 20 mg of lomitapide or a pharmaceutically acceptable salt thereof to be administered in a third dosing period, a daily dose of 40 mg of lomitapide or a pharmaceutically acceptable salt thereof to be administered in a fourth dosing period, and a daily dose of 60 mg of lomitapide or a pharmaceutically acceptable salt thereof to be administered in a fifth dosing period.

In embodiments of any of the kits disclosed herein, the kit may comprise lomitapide dosage units for administration to a pediatric patient aged about 5-10 years, or about 11-15 years, or about 16 to ≤17 years.

In some embodiments, the present disclosure provides a kit for use in any one of the methods disclosed herein, comprising lomitapide dosage units to be administered to a pediatric patient aged about 5-10 years, wherein the dosage units are shown in the Table below. In some embodiments, when the pediatric patient aged about 5-10 years is classified as Child-Pugh A, then the maximum dose is 10 mg.

| Week | Lomitapide Daily Dose (mg) |
|---|---|
| 1 | 2 |
| 4 | 2 |
| +/− 3 days | |
| 8 | 5 |
| +/− 3 days | |
| 12 | 10 |
| +/− 3 days | |
| 16 | 20 |
| +/− 3 days | |

In some embodiments, the present disclosure provides a kit for use in any one of the methods disclosed herein, comprising lomitapide dosage units to be administered to a pediatric patient aged about 5-10 years, wherein the dosage units are shown in the Table below.

| Week | Lomitapide Daily Dose (mg) |
|---|---|
| 1 | 2 |
| 4 | 2 |
| +/− 3 days | |
| 8 | 5 |
| +/− 3 days | |
| 12 | 10 |
| +/− 3 days | |
| 16 | 10 |
| +/− 3 days | |

In some embodiments, the present disclosure provides a kit for use in any one of the methods disclosed herein, comprising lomitapide dosage units to be administered to a pediatric patient aged about 11-15 years, wherein the dosage units are shown in the Table below. In some embodiments, when the pediatric patient aged about 11-15 years is classified as Child-Pugh A, then the maximum dose is 20 mg.

| Week | Lomitapide Daily Dose (mg) |
|---|---|
| 1 | 2 |
| 4 | 5 |
| +/− 3 days | |
| 8 | 10 |
| +/− 3 days | |
| 12 | 20 |
| +/− 3 days | |
| 16 | 40 |
| +/− 3 days | |

In some embodiments, the present disclosure provides a kit for use in any one of the methods disclosed herein, comprising lomitapide dosage units to be administered to a pediatric patient aged about 11-15 years, wherein the dosage units are shown in the Table below.

| Week | Lomitapide Daily Dose (mg) |
|---|---|
| 1 | 2 |
| 4 | 5 |
| +/− 3 days | |
| 8 | 10 |
| +/− 3 days | |
| 12 | 20 |
| +/− 3 days | |
| 16 | 20 |
| +/− 3 days | |

In some embodiments, the present disclosure provides a kit for use in any one of the methods disclosed herein, comprising lomitapide dosage units to be administered to a pediatric patient aged about 16 to ≤17 years, wherein the dosage units are shown in the Table below. In some embodiments, when the pediatric patient aged about 16 to ≤17 years is classified as Child-Pugh A, then the maximum dose is 40 mg.

| Week | Lomitapide Daily Dose (mg) |
|---|---|
| 1 | 5 |
| 4 | 10 |
| +/− 3 days | |
| 8 | 20 |
| +/− 3 days | |
| 12 | 40 |
| +/− 3 days | |
| 16 | 60 |
| +/− 3 days | |

In some embodiments, the present disclosure provides a kit for use in any one of the methods disclosed herein, comprising lomitapide dosage units to be administered to a pediatric patient aged about 16 to ≤17 years, wherein the dosage units are shown in the Table below.

| Week | Lomitapide Daily Dose (mg) |
|---|---|
| 1 | 5 |
| 4 | 10 |
| +/− 3 days | |
| 8 | 20 |
| +/− 3 days | |
| 12 | 40 |
| +/− 3 days | |
| 16 | 40 |
| +/− 3 days | |

The following non-limiting examples illustrate various aspects of the present invention.

### EXAMPLES

### Example 1

The objective of this study was to use determine pediatric starting doses for different age ranges that yield systemic exposures that are similar to those achieved in adults aged 18-55 following a 5mg oral dose using allometric scaling and physiologically-based (PBPK) modeling and simulation approaches.

### Method for Allometric scaling Approach

The first allometric scaling approach for oral clearance in pediatrics is based on the PK relationship:

AUCsteady state=Daily Dose/(CL/F).

Therefore, AUC_{steady state}, adults=Daily Dose_{adults}/(CL/F)_{adults}. AUC_{steady state}, pediatrics=Daily Dose_{pediatrics}/(CL/F)_{pediatrics}. To enable AUC_{steady state, adults}=AUC_{steady state, pediatrics}, Daily Dose_{adults}/(CL/F)_{adults}=Daily Dose_{pediatrics}/(CL/F)_{pediatrics}. Then Daily Dose_{pediatrics}=Daily Dose_{adults}*[(CL/F)_{pediatrics}/(CL/F)_{adults}]. From this equation, it can be seen that the required daily dose in pediatrics is a function of adult dose, adult oral clearance and pediatric oral clearance.

Allometric scaling approach for oral clearance (CL/F) was determined using the following equation

$$CL / F = TV \text{?} \cdot \frac{PNA \text{?}}{PNA \text{?} + 0.31} \cdot ( \frac{BW}{70} ) \text{?}$$

$$\text{?} \text{indicates text missing or illegible when filed}$$

### Method for PBPK Modelling

For the PBPK modeling approach, Simcyp population-based simulator (version 12.2) was used to select the doses for pediatrics age groups that will achieve 80-125% AUC_{geometric mean} and Cmax_{geometric mean} in pediatric subjects (2-18 year old, 50% male) compared to mean AUC and Cmax in adults after 5mg oral dosing (18-55 year old, 50% male). 10 trials were simulated with 20 virtual healthy subjects (adults or children) in each trial. The point estimate and 95% confidence interval around the AUC_{geometric mean} and Cmax_{geometric mean} were compared between different doses. The following statistics were adopted in the analysis: 1) Null hypothesis: point estimates of geometric means for AUC and Cmax between children and adults differ more than 20%, i.e., they are not equivalent (note that the alternative hypothesis is that the point estimates of geometric means for AUC and Cmax in children and adults are equivalent; 2) Statistical test: two-tailed t-test, P=0.05; 3) Variance criteria: 95% confidence interval around the AUCgeometric mean and Cmax,geometric mean, 0.8-1.25 limits. Simcyp estimated human jejunum permeability based on lomitapide physicochemical properties (Peff,man=9.55*10−4 cm/s) was used, which allows for Qgut estimation for each age group that accounts for age-dependent blood flow in intestinal villi that reflects age-dependent cardiac output (14). In the adult group, the estimated Qgut (15.6 L/hr) is similar to previously used fixed Qgut input (17.9 L/hr). The major assumptions made in the model used for simulations were: 1) The fraction of dose absorbed into enterocytes (F(a)) is the same between adults and pediatrics; 2) CYP3A4 accounts for 100% of lomitapide metabolism (elimination) in both children and adults.

### Results

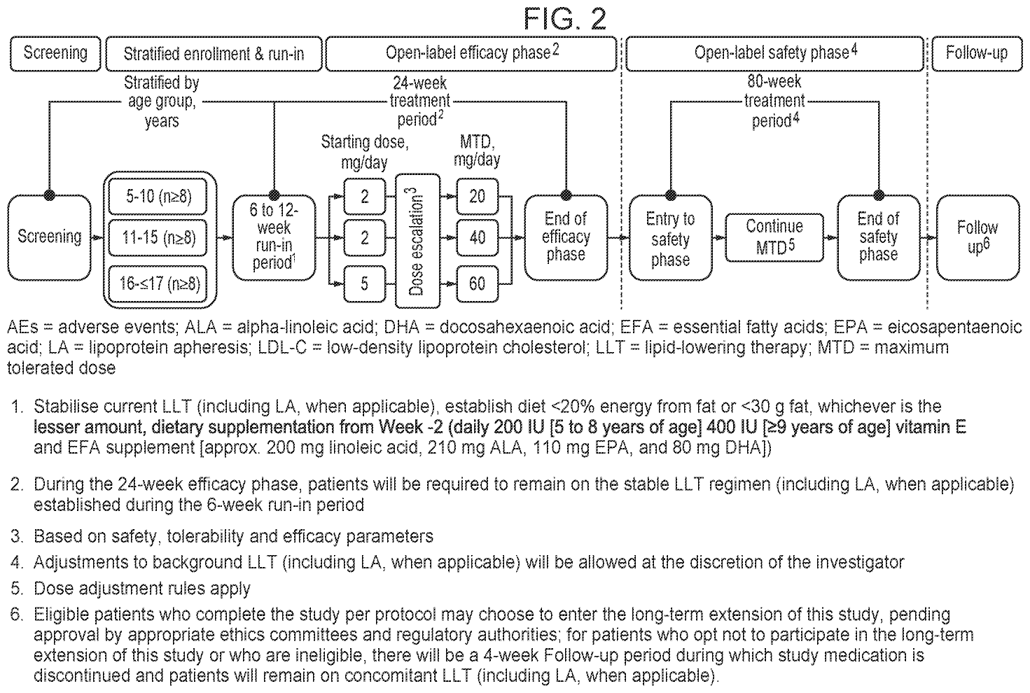

By comparing the calculated doses using both physiologically-based pharmacokinetic (PBPK) modeling and allometric scaling approaches (FIG. 2), the first-in-pediatric doses above 5 years old shown in the table below were proposed.

| Age groups | Suggested starting dose (mg) |
|---|---|
| 5-10 | 2 |
| 11-15 | 2 |
| 16-18 | 5 |

### Example 2

This clinical study is a single-arm, open-label, international, multi-center study to evaluate the efficacy and safety of lomitapide in pediatric patients with Homozygous Familial Hypercholesterolaemia (HoFH) on stable lipid-lowering therapy.

The efficacy of lomitapide is measured by the percent change in low-density lipoprotein cholesterol (LDL-C) at the maximum tolerated dose (MTD) at Week 24±3 days compared to Baseline, when added to stable lipid-lowering therapy (LLT, including lipoprotein apheresis [LA] where applicable) in pediatric patients (5 to ≤17 years of age) with HoFH.

The efficacy of lomitapide in pediatric HoFH patients is also assessed by:

Percent change from Baseline at Week 24±3 days in lipid parameters, including total cholesterol (TC), non high-density lipoprotein cholesterol (Non-HDL-C), very low-density lipoprotein cholesterol (VLDL-C), triglycerides (TG), lipoprotein(a) [Lp(a)], and apolipoprotein B (apo B);

Percent change from Baseline in TC, non-HDL-C, LDL-C, TG, VLDL-C, Lp(a), and apo B at all other time-points through Week 104±1 week;

Change in LLT and LA from Week 24±3 days through Week 104±1 week; and

Number (percent) of pediatric HoFH patients achieving the European Atherosclerosis Society (EAS) recommended target LDL-C of <135 mg/dL (3.5 mmol/L) at Week 24±3 days and at any time on study.

### Abbreviations

| Term or | |
|---|---|
| Abbreviation | Description |
| AE | Adverse event |
| ALA | Alpha-linolenic acid |
| ALT | Alanine transaminase |
| AP | Alkaline phosphatase |
| apo A-I | Apolipoprotein A-I |
| apo B | Apolipoprotein B |
| ASCVD | Atherosclerotic cardiovascular disease |
| AST | Aspartate transaminase |
| ATC | Anatomical Therapeutic Chemical |
| BMI | Body mass index |
| BG | Blood glucose |
| BSA | Body surface area |
| BUN | Blood urea nitrogen |
| CBC | Complete blood count |
| CK | Creatinine kinase |
| Cₘₐₓ | Maximum concentration |
| CVD | Cardiovascular disease |
| CYP | Cytochrome P 450 |
| DHA | Docosahexaenoic acid |
| EAS | European Atherosclerosis Society |
| ECG | Electrocardiogram |
| eCRF | Electronic Case Report Form |
| EFA | Essential fatty acids |
| EMA | European Medicines Agency |
| EPA | Eicosapentaenoic acid |
| EoT | End of Treatment |
| FDA | Food and Drug Administration |
| FH | Familial Hypercholesterolaemia |
| GGT | Gamma-glutamyl transferase |
| GFR | Glomerular filtration rate |
| HBsAg | Hepatitis B surface antigen |
| Anti-HBC | Antibody to Hepatitis C |
| HDL-C | High-density lipoprotein cholesterol |
| HMG-COA | 3-hydroxy-3-methylglutaryl coenzyme A |
| HoFH | Homozygous familial hypercholesterolaemia |
| IU | International units |
| LA | Lipoprotein apheresis |
| LDL | Low-density lipoprotein |
| LDL-C | Low-density lipoprotein cholesterol (conventional |
| | mg/dl units can be converted to SI [mmol/L] units |
| | using the factor 0.0259) |
| LDLR | LDL receptor |
| LDLRAP1 | LDL-receptor adapter protein 1 |
| LLT | Lipid-lowering therapy (including LA, where applicable) |
| Lp(a) | Lipoprotein a |
| LTE | Long-term extension |
| MTD | Maximum tolerated dose |
| MTP | Microsomal triglyceride transfer protein |
| Non-HDL-C | Non-high-density lipoprotein cholesterol |
| NYHA | New York Heart Association |
| PCSK9 | Proprotein convertase subtilisin/kexin type 9 |
| PK | Pharmacokinetics |
| SmPC | Summary of Product Characteristics |
| TC | Total cholesterol |
| TG | Triglycerides |
| ULN | Upper limit of normal |
| VLDL-C | Very low density lipoprotein cholesterol |
| WHO | World Health Organization |
| MTD | Maximum tolerated dose |
| MTP | Microsomal triglyceride transfer protein |
| Non-HDL-C | Non-high-density lipoprotein cholesterol |
| NYHA | New York Heart Association |
| PCSK9 | Proprotein convertase subtilisin/kexin type 9 |
| PK | Pharmacokinetics |
| SmPC | Summary of Product Characteristics |
| TC | Total cholesterol |
| TG | Triglycerides |
| ULN | Upper limit of normal |
| VLDL-C | Very low density lipoprotein cholesterol |
| WHO | World Health Organization |

### Inclusion Criteria

Patients eligible for participation include all of the following criteria :
1. Male and female patients aged 5 to ≤17 years with HoFH as defined by any of the following criteria recommended by the Consensus Panel on Familial Hypercholesterolaemia of the EAS (Cuchel, Bruckert et al. 2014):
   Genetic confirmation of 2 mutant alleles at the LDLR, apo B, PCSK9, or LDLRAP1 gene locus OR
   An untreated LDL-C >500 mg/dL (13 mmol/L) or treated LDL-C ≥300 mg/dL (8 mmol/L) together with either
      Cutaneous or tendon xanthoma before age 10 years or
      Untreated LDL-C levels consistent with heterozygous FH in both parents
2. Baseline LDL-C on LLT (Cmax of LDL-C immediately prior to LA, ifapplicable)
   >160 mg/dL (4.1 mmol/L, no documented CVD) or
   >130 mg/dL (3.4 mmol/L, established CVD defined as aortic valve disease and/or coronary atherosclerosis)
3. Body weight ≥15 kg or BMI and height both >10^{th} percentile according to WHO Growth Charts for Boys and Girls 5 to 19 Years of Age (see WHO Growth Charts)
4. Patient and/or his/her legal representative are informed, have read and understood the patient information/informed consent form, and have given written informed assent/consent
5. Patient and/or his/her legal representative follow study procedures and instructions, particularly that
   LLT (including LA when applicable) is stable for at least 6 weeks prior to Baseline (Run-in Period) and remains stable through Week 24±3 days (end of Efficacy Phase)
   The patient is compliant with both the low-fat diet supplying <20% of energy (calories) from fat or <30 g fat, whichever is the lesser amount starting at the beginning of the Run-in Period and the dietary supplement regimen starting at Week—2 of the Run-in Period, both continuing until completion of the study (and the LTE of this study, when applicable)
6. Postmenarchal female adolescents use an effective form of birth control with failure rates <1% per year (e.g., implant, injectable, combined oral contraceptive, intrauterine contraceptive device, sexual abstinence, vasectomy or vasectomised partner) during participation in the study (and at least 4 weeks thereafter). Patients taking oestrogen-based oral contraceptives are advised about possible loss of effectiveness due to diarrhea and/or vomiting. Additional contraceptive measures are used for 7 days after resolution of symptoms.
7. Patient must be in stable physical and mental health at screening

### Exclusion Criteria

Patients are not included if any of the following criteria apply:
1. Other forms of primary hyperlipoproteinaemia and secondary causes of hypercholesterolaemia (e.g., nephrotic syndrome, hypothyroidism)
2. Contraindications for the use of lomitapide according to section 4.3 of the EMA Summary of Product Characteristics (SmPC), such as hypersensitivity to the active substance or to any of the excipients listed in Section 6.1 of the SmPC, known significant or chronic inflammatory bowel disease or malabsorption
3. Moderate (Child-Pugh B) or severe hepatic impairment (Child-Pugh C), active liver disease and/or abnormal liver function tests at screening (AST or ALT>1.5×ULN and/or total bilirubin >1.5×ULN in the absence of Gilbert's syndrome or AP>1.5×ULN [based on appropriate age and gender normal values])
4. Serum CK>2×ULN
5. Chronic renal insufficiency with glomerular filtration rate (GFR) <70 mL/min/1.73 m² calculated using the Schwartz formula
6. Uncontrolled hypertension (defined as mean systolic and/or diastolic blood pressure ≥95% of normal for age and sex) despite medical therapy
7. New York Heart Association (NYHA) Class III or IV congestive heart failure
8. Precocious/delayed puberty or endocrine disorder affecting growth (e.g., hypothyroidism, premature adrenarche)
9. History of drug abuse within the last 3 years or habitual alcohol consumption (defined as >1 ounce [28 g] of liquor or 4-ounce glass [113 g] of wine, or the equivalent, ≥3 times per week)
10. Life expectancy predicted to be <5 years
11. History of a non-skin malignancy (with the exception of cervical cancer in situ) within 3 years prior to enrolment
12. Treatment with any Investigational Medicinal Product (IMP) within 6 months or 5 times the terminal half-life of the corresponding IMP, whichever is longer, before the screening visit
13. Patient is a dependent of the sponsor, of the investigational team or his/her immediate family
14. Pregnant or nursing women

### Outcomes

The primary efficacy endpoint is:

Percent change in LDL-C at Week 24±3 days compared to Baseline

### Secondary Endpoints

The secondary efficacy endpoints of this study are:
Percent change from Baseline at Week 24±3 days for the following lipid parameters: TC, Non-HDL-C, VLDL-C, TG, Lp(a), and apo B
Percent change from Baseline at all other time points through Week 104±1 week for the following lipid parameters: LDL-C, TC, Non-HDL-C, VLDL-C, TG, Lp(a), and apo B
Change in LLT and LA from Week 24±3 days through Week 104±1 week
Total number and percent of patients achieving the EAS recommended target LDL-C of <135 mg/dL (3.5 mmol/L) in pediatric HoFH patients at Week 24±3 days and at any time on study

### Statistical Methods

The key efficacy and safety analyses are conducted once all patients have completed or withdrawn prior to Visit 10 at Week 24±3 days (End of the Efficacy Phase) upon entry and cleaning of all data including Visit 10 in order to allow for an early submission of an application for regulatory approval of Lojuxta® in the pediatric indication. All descriptive statistics of the efficacy parameters and all analyses of safety data collected during the Safety Phase are performed after all data up to the FU Visit 23 (Week 108) has been entered and cleaned (including AE and concomitant medication data with date of onset until FU visit [Week 108]).

### Study Medications

### Formulation, Packaging and Labelling

The investigational product is supplied in high-density polyethylene bottles with polyester, aluminium foil, and cardboard induction seals and child-resistant polypropylene screw caps. It is packed and labelled according to applicable regulatory requirements.

Lomitapide is prepared as a crystalline methanesulfonate salt. The investigational product is administered orally in opaque, hard gelatin capsules. Four bottle types contain 28 capsules each by 2 mg, 5 mg, 10 mg, and 20 mg strength. Each hard capsule contains lomitapide mesylate equivalent to the labeled content of the free-base form of lomitapide, and the following inactive excipients: pregelatinised starch (maize), sodium starch glycolate (Type A), microcrystalline cellulose, lactose monohydrate, colloidal, anhydrous silica, and magnesium stearate.

### Drug and Dietary Supplements Accountability

A sufficient number of lomitapide bottles are provided to sites in bulk at appropriate intervals depending on the phase of the study and accrual of patients. Dietary supplements are sourced locally, where possible. The investigator, or the person designated by the investigator, are responsible for dispensing the appropriate bottle or combination of bottles to a patient at each study visit. At each site, the investigator, or the person designated by the investigator, is responsible for keeping accurate records of study medication accountability comprising the receipt, the dispensing, and the return of all used and partially unused study medication throughout the study. The drug accountability form is kept up to date and will be reviewed periodically by the study monitor. Patients are asked to keep all used and partially unused bottles of study medication and return them to the site during the scheduled visits. For drug accountability, the number of used and partially unused bottles and unused capsules are counted. Compliance with dietary supplements is discussed with the patients at each visit and is documented in the electronic Case Report Form (eCRF). It is also verified by reviewing the patient Diet Records. After drug accountability is completed, all unused or partially used study medication are returned to the sponsor or disposed of by the study site. The study monitor instructs the site on the return of all study medication. A final inventory of the total amount of study medication and dietary supplements at each study site against the amount used and returned is recorded. Inventory records are readily available for inspection by the study monitor and/or auditor, and open to government inspection at any time.

### Dietary Supplements

Patients are administered daily oral supplementation with vitamin E (200 IU for patients 5 to 8 years of age, 400 IU for patients 9 to ≤17 years of age) and an EFA supplement containing approximately 200 mg linoleic acid, 210 mg ALA, 110 mg EPA, and 80 mg DHA. Dietary supplements is dispensed during Visit 3 at Week −3 to Week −2 (Run-in Compliance), during Visit 4 at DO (Baseline, Efficacy Phase), and at every visit thereafter until Visit 21 at Week 92±1 week.

### Diet Instructions and Diet Records

The occurrence and severity of GI AEs associated with the use of lomitapide decreases in the presence of a low-fat diet. Patients start to follow a diet supplying <20% of energy from fat or <30 g fat, whichever is the lesser amount at Visit 2, the Start of the Run-in Period, and continue this diet until the EoT (and the LTE of this study, when applicable).

A dietitian provides dietary counselling to the patients and their parents/legal guardians from Visit 2 (Start of the Run-in Period) through Visit 5 at Week 4±3 days. Patients and parents/legal guardians are instructed on how to consume <20% energy from fat or <30 g fat, whichever is the lesser amount. Instructions include detailed information concerning the role of dietary fat and GI-related symptoms and patients are encouraged to pay special attention to foods they eat when/if they experience these symptoms. The dietitian reviews current eating habits in order to minimize adverse events and address potential adherence problems. The dietitian alsotailors to each patient's caloric needs toensure ahealthy weight and normal growth is maintained.

The dietitian also reviews compliance with the diet and discusses any issues or concerns the patient and his/her parents/legal guardians may have with the diet at Visit 3 (Week −3 to Week −2). The dietitian informs patients and their parents/legal guardians to call him/her to discuss over the phone or schedule a meeting to address any diet-related questions or concerns any time during the study. Additional resources pertaining to the diet are available to the dietitian and the patient and his/her parent/legal guardian.

Weight is measured using a consistent approach at Screening and at each visit after the Run-in Period (i.e., child/adolescent always weighed in same amount of clothing, either in underwear, hospital gowns or by subtracting the weight of clothes). Patients of normal or below normal body weight with weight loss >3% since the last visit are instructed by the dietitian on how to increase caloric intake based on individual needs. The standard recommended fat intake for children >2 years old is to follow a diet with <30% energy from fat, hence, decreasing dietary fat intake to <20% or <30 g fat, whichever is the lesser amount should not pose any risk to patients of any age that may be enrolled in this study.

The investigator requests additional dietary counselling as clinically indicated. At the same visits, 2-Day Diet Records are given to the patients and their parents/legal guardians.

In order to assess dietary compliance and to provide information for interpreting possible GI AEs, patients are instructed to document all food and beverages consumed periodically throughout the study, using Diet Records. A 2-Day Diet Record is dispensed and returned from Visit 2 (Start of the Run-in Period) through Visit 21 at Week 92±1 week.

When diet-related problems are noted during the study, e.g., weight loss or GI-related complaints such as diarrhoea, the patients are requested to fill out additional Diet Records. Note that Diet Records collected during the Run-in Period are not analysed, but are used by the dietitian on site to assess compliance before beginning lomitapide treatment.

Patients treated with lomitapide and their parents/legal guardians are advised of the potential risk of dehydration in relation to GI AEs and take precautions to avoid fluid depletion.

### Study Design

This is a single-arm, open-label, multi-centre phase III study to evaluate the efficacy and long-term safety of lomitapide in pediatric patients with HoFH receiving stable LLT (including LA, when applicable). Each patient participates for up to 120 weeks (about 2.5 years) in the study (see FIG. 2).

The study consists of 5 periods (see FIG. 2):
1. Screening Period (starting at Week −12, i.e. ≤12 weeks prior to Baseline for up to 6 weeks)
2. Stratified Enrolment and Start of Run-in Period (starting at minimum at Week −6, i.e., 6 weeks prior to Baseline for a minimum of 6 weeks):
   Enrolment is stratified to ensure approximately equal numbers of patients in the following age groups: 5 to 10 years, 11 to 15 years, and 16 to ≤17 years (with ≥8 patients in any individual age group).
   Patients are stabilised on current LLT (including LA, when applicable) and established on a diet supplying <20% of energy (calories) from fat or <30 g fat, whichever is the lesser amount.
   Daily supplementation with vitamin E (200 international units [IU] for patients 5 to 8 years of age, 400 IU for patients 9 to ≤17 years of age) and an EFA supplement containing approximately 200 mg linoleic acid, 210 mg ALA, 110 mg EPA, and 80 mg DHA starting at Week −2
3. Efficacy Phase (starting at Baseline, i.e. Day [D] 0 for 24 weeks±3 days)
   Approximately 45 pediatric patients with HoFH are treated with lomitapide given orally, added to their current, stable LLT (including LA, when applicable) established during the Run-in Period
   Assuming a withdrawal rate of approximately 33% by Week 24±3 days, this results in 30 evaluable patients at Week 24±3 days (with ≥8 patients in any individual age group).
   After the stabilization of the patient on his/her current MTh of LLT (including LA, when applicable) during the 6-week Run-in Period, treatment with lomitapide is started as an add-on therapy on DO of the Efficacy Phase.
   Dosing is initiated at the recommended starting dose and escalated to the maximum dose as applicable to the age groups based upon safety and tolerability in addition to LDL-C values.
   The first dose of study medication is administered at the study site on D0.
   During the 24-week Efficacy Phase, patients are required to remain on the stable LLT regimen (including LA, when applicable) established during the 6-week Run-in Period
4. Safety Phase (starting at Week 24±3 days for 80±1 weeks)
   Patients enter the 80-week Safety Phase after the Week 24±3 days assessments have been completed. Each patient continues receiving the MTD of lomitapide he/she achieved during the Efficacy Phase (unless criteria is met for reducing the dose) for an additional 80±1 weeks in the Safety Phase.
   When after Week 24±3 days, a patient has crossed over into the next age category, the study medication is escalated to the maximum dose applicable for the new age category. When the patient tolerates this new dose for ≥4 weeks, then this is considered the new MTD.
   As necessary during the 80-week Safety Phase, the lomitapide dose is reduced from the MTD due to tolerability or safety issues, and the patient is re-challenged after a minimum period of 4 weeks following dose reduction with a higher dose of lomitapide once these issues resolve, but the dose during the Safety Phase does not exceed the MTD established during the Efficacy Phase.
   Adjustments to background LLT (including LA, when applicable) are allowed at the discretion of the investigator.
5. Follow-up (starting at Week 104±1 week for 4 weeks) or participation in the LTE of this study
   At Week 104±1 week, eligible patients who complete the study per protocol can enter the LTE of this study, pending approval by appropriate ethics committees and regulatory authorities. A separate Protocol Amendment is prepared describing the LTE in detail.

For patients who opt not to participate in the LTE of this study or who are ineligible, there is a 4-week Follow-up period during which study medication is discontinued and patients remain on concomitant LLT (including LA, when applicable)

### Interventions

This is an open-label study, blinding of treatment is not applicable.

After the stabilization of the patient on his/her current MTD of LLT (including LA when applicable) during the 6-week Run-in Period, treatment with lomitapide is started as add-on therapy on D0 of the Efficacy Phase.

Lomitapide capsules is provided in 4 dose strengths of 2 mg, 5 mg, 10 mg, and 20 mg. Dosing is initiated at the recommended starting dose and escalated to the maximum dose as applicable to the age groups (see Table 1 and FIG. 2) based upon safety and tolerability in addition to LDL-C values.

TABLE 1
Lomitapide Starting Dose and Dose Escalation by Age Group

| Age | Lomitapide Dose (mg) | | | | | |
|---|---|---|---|---|---|---|
| Group | | Week 4 | Week 8 | Week 12 | Week 16 | |
| (years) | D0 | ±3 days | ±3 days | ±3 days | ±3 days | Maximum |
| 5 to 10 | 2 | 2 | 5 | 10 | 20 | 20 (10, in |
| | | | | | | Child-Pugh A) |
| 11 to 15 | 2 | 5 | 10 | 20 | 40 | 40 (20, in |
| | | | | | | Child-Pugh A) |
| 16 to ≤17 | 5 | 10 | 20 | 40 | 60 | 60 (40, in |
| | | | | | | Child-Pugh A) |

Each patient takes 1 to 3 capsule(s) once daily to achieve the doses specified in the titration scheme. The first dose of study medication is administered at the study site on D0.

As of D1, patients self-administer (or the patient's parent/legal guardian administers to the patient) lomitapide orally, once daily, at approximately the same time each day. Ideally, lomitapide is administered at least 2 hours after the evening meal (e.g., at bedtime) with a glass of water on an empty stomach because the fat content of a recent meal may adversely impact GI tolerability. Patients who are unable to swallow the intact capsule(s) open the capsule(s) and sprinkle the capsule content on 1 tablespoon of apple sauce or mashed banana, which are fat-free.

In case of a missed dose, patients are instructed to take missed doses of lomitapide only if they can be taken at least 12 hours prior to the next scheduled dose. Dietary supplements are ideally taken in the morning. Patients taking bile acid sequestrants are reminded that they should take lomitapide 4 hours before or 4 hours after this class of medications. If atorvastatin is given concomitantly, lomitapide is administered 12 hours apart.

1-7. (canceled)

8. A method of treating homozygous familial hypercholesterolemia (HoFH) in a pediatric patient in need thereof, comprising:
(a) administering an initial dose of 2 mg per day of lomitapide, or a pharmaceutically acceptable salt thereof, to a patient aged 5 to 10 years for about 4 to about 8 weeks;
(b) administering an initial dose of 2 mg per day of lomitapide, or a pharmaceutically acceptable salt thereof, to a patient aged 11 to 15 years for about 1 to about 4 weeks; and
(c) administering an initial dose of 5 mg per day of lomitapide, or a pharmaceutically acceptable salt thereof, to a patient aged 16 to 17 years for about 1 to about 4 weeks.

9. The method of claim 8, wherein the administration provides a reduction in the patient's low-density lipoprotein (LDL-C) compared to prior to the administration.

10. The method of claim 8, wherein the administration provides a reduction in the patient's LDL-C to less than 135 mg/dL.

11. The method of claim 8, wherein the administration provides a reduction in the patient's total cholesterol (TC), apolipoprotein B (apo B), non-high-density lipoprotein cholesterol (non-HDL-C), very-high dense lipoprotein cholesterol (VLDL-C), triglyceride, and high-density lipoprotein cholesterol (HDL-C) compared to prior to the administration.

12. The method of claim 8, further comprising titrating the initial dose to a maximum daily dose of 20 mg per day for the patient aged 5 to 10 years.

13. The method of claim 8, further comprising titrating the initial dose to a maximum daily dose of 40 mg per day for the patient aged 11 to 15 years.

14. The method of claim 8, further comprising titrating the initial dose to a maximum daily dose of 60 mg per day for the patient aged 16 to 17 years.

15. The method of claim 8, further comprising titrating the initial dose to a second dose, wherein the second dose is:
(a) 5 mg per day of lomitapide, or pharmaceutically acceptable salt thereof, for the patient aged 5 to 10 years,
(b) 5 mg per day of lomitapide, or pharmaceutically acceptable salt thereof, for the patient aged 11 to 15 years, and
(c) 10 mg per day of lomitapide, or pharmaceutically acceptable salt thereof, for the patient aged 16 to 17 years,
wherein the second dose is administered for about 1 to about 4 weeks.

16. The method of claim 15, further comprising titrating the second dose to a third dose, wherein the third dose is:
(a) 10 mg per day of lomitapide, or pharmaceutically acceptable salt thereof, for the patient aged 5 to 10 years,
(b) 10 mg per day of lomitapide, or pharmaceutically acceptable salt thereof, for the patient aged 11 to 15 years, and
(c) 20 mg per day of lomitapide, or pharmaceutically acceptable salt thereof, for the patient aged 16 to 17 years,
wherein the third dose is administered for about 1 to about 4 weeks.

17. The method of claim 16, further comprising titrating the third dose to a fourth dose, wherein the fourth dose is:
(a) 20 mg per day of lomitapide, or pharmaceutically acceptable salt thereof, for the patient aged 5 to 10 years,
(b) 20 mg per day of lomitapide, or pharmaceutically acceptable salt thereof, for the patient aged 11 to 15 years, and
(c) 40 mg per day of lomitapide, or pharmaceutically acceptable salt thereof, for the patient aged 16 to 17 years,
wherein the fourth dose is administered for about 1 to about 4 weeks.

18. The method of claim 8, wherein the patient has hepatic impairment characterized by Child-Pugh A.

19. The method of claim 18, further comprising titrating the initial dose to a maximum daily dose of 10 mg per day for the patient aged 5 to 10 years.

20. The method of claim 18, further comprising titrating the initial dose to a maximum daily dose of 20 mg per day for the patient aged 11 to 15 years.

21. The method of claim 18, further comprising titrating the initial dose to a maximum daily dose of 40 mg per day for the patient aged 16 to 17 years.

22. The method of claim 8, wherein the patient is on a stable lipid-lowering therapy.

23. The method of claim 8, further comprising administering to the patient vitamin E, alpha-linolenic acid, linoleic acid, eicosapentaenoic acid, and docosahexaenoic acid.

24. The method of claim 8, wherein the lomitapide or pharmaceutically acceptable salt thereof is a lomitapide mesylate.

25. The method of claim 24, wherein the lomitapide mesylate is encapsulated in a capsule.

26. The method of claim 8, wherein the patient has at least 2 mutant alleles at the LDLR, apo B, PCSK9, or LDLRAP1 gene locus.

27. The method of claim 8, wherein the patient has an untreated LDL-C level of greater than 500 mg/dL and cutaneous or tendon xanthoma before 10 years of age or untreated LDL-C level consistent with heterozygous familial hypercholesterolemia in both parents.

28. The method of claim 8, wherein the patient has a treated LDL-C level of at least 300 mg/dL and cutaneous or tendon xanthoma before 10 years of age or untreated LDL-C level consistent with heterozygous familial hypercholesterolemia in both parents.

29. The method of claim 8, wherein the patient has no documented cardiovascular disease and LDL-C level of greater than 160 mg/dL prior to the administration.

30. The method of claim 8, wherein the patient has established cardiovascular disease and LDL-C level of greater than 130 mg/dL prior to the administration.
